(19) United States
(12) Patent Application Publication
(10) Pub. No.: US 2002/0122780 A1
(43) Pub. Date: Sep. 5, 2002

(54) Photochromic hair coloring composition

(76) Inventors: Marjorie McManus, Bloomfield, NJ (US); Blazena Federer, Waldwick, NJ (US)

Correspondence Address: Pendorf & Cutliff, Tampa Office, P O Box 20445, Tampa, FL, 33622-0445, US

(21) Appl. No.: 09/549,750

(22) Filed: Apr. 14, 2000

(51) Int. Cl. A61K 7/06

(52) U.S. Cl. 424/70.6; 424/70.1

(57) ABSTRACT

A photochromic hair coloring composition and a method for applying to hair a photochromic composition, for example, selectively to form a design, which is not visible indoors but becomes visible upon exposure to sunlight.

### BACKGROUND OF THE INVENTION

1. Field of the Invention

The present invention concerns a hair coloring composition, and more specifically, a photochromic hair coloring composition, and a method for applying to hair a pattern which is not visible indoors but becomes visible upon exposure to sunlight.

2. Description of the Related Art

Recently, photochromic materials have been introduced into some topical cosmetic products. Basically, particles containing photochromic pigment are simply mixed into a cosmetic cream or ointment type base. Such compositions are formulated to be easily applied in the morning and easily wiped off in the evening. For example, U.S. Pat. No. 5,176,905 (Ohno, et al.) entitled “Photochromic flesh-colored pigment and process for producing the same” teaches a photochromic flesh-colored pigment treated to be hydrophobic, with good dispersibility and sufficient photochromism. Indicated uses of the product include lipstick, rouge and mascara. U.S. Pat. No. 5,762,913 (Tanaka, et al.) entitled “Photochromic compound oxide and cosmetic comprising the same” also teaches cosmetic compositions.

U.S. Pat. No. 4,929,693 (Akashi, et al.) entitled “Photochromic compound” teaches photochromic polymer particles having an average particle size of 0.1 μm to 100 μm. If the average particle size is less than 0.1 μm, the cosmetic may have a problem with respect to its safety. If the average particle size is more than 100 μm, the efficiency of the photochromic reaction is reduced, so that the color density in the colored state may be lowered. A cosmetic containing these polymer particles can effectively protect the human skin from ultraviolet light, and the cosmetic shows color change to present a fashionable appearance.

Hair styling is a popular way to make a statement. Individuals express themselves through the cut, arrangement and color of their hair. However, unlike the cosmetics mentioned above, hair color change is intended to be relatively permanent. Hair colorants must have good resistance to, e.g., washing, brushing, bad weather, and perspiration. In one of the conventional methods of changing hair color, oxidation dyes are permeated into hair in an alkaline condition, treated with oxidizing agents such as hydrogen peroxide or the like and then are polymerized within the hair for developing color. Alternatively, acid dyes are permeated into the hair for dyeing.

It would be interesting to provide a hair coloring composition that is photochromic, such that hair treated with such a composition appears normal while the individual is at work in an office or in school, but exhibits an altered coloration while the individual is in the sun or under and ultraviolet radiation source.

### SUMMARY OF THE INVENTION

It is therefore an object of the present invention to provide a novel commercially acceptable photochromic hair coloring composition.

It is a further object of the invention to provide a method of treating hair to render the treated hair photochromic.

These objectives are achieved in a first embodiment of the invention by a photochromic hair coloring composition comprising photochromic monomers (such as spiropyran or spiroxazine molecules), a carbopol gel matrix, and sufficient rapid-drying dispersant (e.g., about 25% ethanol) to help disperse the photochromic slurry.

The objectives are achieved in a second embodiment of the invention by a hair coloring composition comprising anti-hair antibodies immobilized on photochromic pigment particles. Since such particles provide a good and strong coloring capability and are specific to hair, such a hair coloring composition will neither stain skin nor cause skin or eye irritation. Further, the touch and feel of the hair will be improved.

The photochromic hair coloring composition, if in liquid form, can be applied to selected areas of the hair by an applicator or hair wand similar to a regular eye mascara wand. The wand would make it possible to apply one or more photochromic materials to selective areas of the hair, such that multicolor words, symbols or pictures are hidden while the individual is indoors, and become rapidly visible under direct sunlight.

In order to enhance the photochromic effect, darkly colored hair is preferably bleached to a lighter color prior to treating with photochromic hair coloring composition.

The photochromic hair coloring composition is capable of changing from clear to any visible color (violet, red, blue, yellow, etc.) in presence of sunlight or any ultraviolet radiation source. Hair treated in accordance with one embodiment of the present invention will revert to colorless as soon as it is removed from the sunlight or the UV source. Hair treated in accordance with the present invention may be capable of undergoing multiple reversible changes without altering its physical properties.

In addition to the novel color changing feature of this invention, the photochromic hair coloring composition has the ability to act as a filter of harmful UV rays. Spiroxazine and spiropyran molecules absorb UV radiation between 280 and 400 nm (cf., U.S. Pat. No. 5,581,090) allowing hair treated with the inventive photochromic coloring composition to act as a UV block, thus protecting the head from the harmful effects of UV radiation.

The foregoing has outlined rather broadly the features and technical advantages of the present invention in order that the detailed description of the invention that follows may be better understood. Additional features and advantages of the invention will be described hereinafter which form the subject of the claims of the invention.

### DETAILED DESCRIPTION OF THE INVENTION

First Embodiment—Composition Containing Photochromic Monomers

In a first embodiment of the invention, the photochromic hair coloring composition comprises photochromic monomers, preferably microencapsulated, a gel matrix/suspending agent (preferably a Carbopol™ gel matrix), and a dispersing agent such as ethanol (about 25) to help disperse the photochromic slurry.

Photochromic monomers which can be used in the present invention include any reversible photochromic compounds as employed in this art which retain photochromaticity after incorporation in the matrix or base, and specific examples include napthopyran compounds and other photochromic compounds as taught in U.S. Pat. Nos. 5,721,059; 5,558,700; 5,558,699; 5,514,635; 5,502,967; 5,458,815; 5,458,814; 5,466,398; 5,384,077; 5,451,344; 5,429,774; 5,411,679; 5,441,418; 5,405,958; 5,381,193; 5,369,158; 5,340,857; 5,274,132; 5,244,602; 5,223,958; 4,679,918; 4,556,605; and 4,498,919, the disclosures of these patents being incorporated herein by reference.

Specific examples of photochromic material which can be used in the present invention include organic photochromic substances such as azobenzene compounds, thioindigo compounds, dithizone metal complex compounds, spiropyran compounds, spirooxazine compounds, fulgide compounds, dihydropyrene compounds, spirothiopyran compounds, 1,4-2H-oxazine, triphenylmethane compounds, viologen compounds, naphthopyran compounds, benzopyran compounds and so on. Particularly preferred for purposes of the invention are spiropyran compounds, spirooxazine compounds, fulgide compounds, naphthopyran compounds and benzopyran compounds.

Among specific examples of photochromic substances are 1,3,3-trimethylspiro[indoline-2,3′-(3H)naphtho(2,1-b)(1,4) -oxazine], 6′-indolino-1,3,3-trimethylspiro[indoline-2,3′-(3H)naphtho(2,1-b)(1,4)-oxazine], 5-chloro-1,3,3-trimethylspiro[indoline-2,3′-(3H)naphtho(2,1-b)(1,4)-oxazine], 6′-piperidino-1,3,3-trimethylspiro[indoline-2,3′-(3H)naphtho(2,1-b)(1,4)-oxazine], 1-benzyl-3,3-dimethylspiro[indoline-2,3′-(3H)naphtho(2,1-b)(1,4)-oxazine], 1,3,5,6-tetramethyl-3-ethylspiro[indoline-2,3′-(3H)naphtho(2,1-b)(1,4)-oxazine], 1,3,3,5,6-pentamethylspiro[indoline-2,3′-(3H)naphtho(2,1-b)(1,4)-oxazine], 1,3,5,6-tetramethyl-3-ethylspiro[indoline-2,3′-(3H)pyrido(3,2-f)(1,4)-benzoxazine], 1,3′,3′-trimethylspiro(2H-2-benzopyran-2,2′-indoline), 1,3,3-triphenylspiro[indoline-2,3′-(3H)naphtho (2,1-b) pyran], 1-(2,3,4,5,6-pentamethylbenzyl)-3,3-dimethylspiro[indoline-2,3′-(3H)naphtho(2,1-b)pyran], 1-(2-nitrobenzyl)-3,3-dimethylspiro[indoline-2,3′-(3H)naphtho(2,1-b)pyran], 2,2-diphenylnaphtho(2,1-b)pyran, 2,2-di(p-methoxyphenyl)naphtho(2,1-b)pyran, 2,5-dimethylfuryltrimethylfulgide, 2-methyl-5-chlorotrimethylfulgide, spiro[2H-chromen-2,2′-tricyclo[3.3.1.1.-sup.3.7]decane, spiro[2H-naphtho[1,2-b]pyran-2,2′-tricyclo[3.3.1.1.sup.3.7]decane], 5,7-dimethylspiro[2H-chromene-2,2′-tricyclo[3.3.1.1.sup.3.7]decane], 6-(4-methoxyphenyl)-9-methoxyspiro[2H-naphtho[1,2-b]pyran-2,2′-tricyclo][3.3.1.1.sup.3.7]decane], 6-chlorospiro[2H-naptho[1,2-b]pyran-2,2′-tricyclo[3.3.1.1.sup.3.7] decane and so on.

The photochromic substances can be used independently but for the purpose of improving the color changing function or the fastness to light. It is preferable to concomitantly use auxiliary agents such as high-boiling solvents, plasticizers, synthetic resins, hindered amines, hindered phenols and so on. These compounds are well known additives for use in combination with photochromic substances and their proportions can be selected from the known ranges. In the present invention, for the purpose of improving the light fastness of said organic photochromic substances, the use of a hindered amine compound or a combination of a hindered amine and other auxiliary agents is recommended.

The suspending agent can be any composition which aids in bonding the photochromic molecules (or, in the case that pigment particles are used, pigment particles) to hair, yet does not significantly adversely effect the touch and feel of hair. Suspending agents may be selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives. Polyacrylic acid is available commercially as Carbopol™ 420, Carbopol™ 488 or Carbopol™ 493. Polymers of acrylic acid cross-linked with a polyfunctional agent may also be used, they are available commercially as Carbopol™ 910, Carbopol™ 934, Carbopol™ 940, Carbopol™ 941 and Carbopol™ 980. An example of a suitable copolymer of a carboxylic acid containing a monomer and acrylic acid esters is Carbopol™ 1342. All Carbopol™ materials are available from Goodrich and Carbopol™ is a trademark.

Suitable cross-linked polymers of acrylic acid and acrylate esters are Pemulen TR1 or Pemulen TR2. A suitable heteropolysaccharide gum is xanthan gum, for example that available as Kelzan mu.

One specific carbopol gel could comprise water, PVP K30, Carbopol™ 940, triethanolamine, glydant, ploysorbate 20, and fragrance. The carbopol gel could also contain a preservative (e.g., propylene glycol, propyl paraben, methyl paraben), an emollient (glyceryl monostearate, aloe vera gel), an emulsifier (stearic acid), an anti-oxidant (vitamin E acetate). Further optional ingredients include 1,2,3 trihydroxypropane, 4-paraminobenzoic acid, 2-hydroxy-4-methoxyl-phenyl, 2-amino 3-p-hydroxyphenyl-propanoic acid, hydroxyethane, 1-hexadecanol, 7-dehydrocholesterol, carbopol gel, miglyol-gel-B, octyldimethyl PABA, d-Panthenol, glyceryl, dimethylpolysiloxane, petrolatum, mineral oil, jojoba oil, mink oil, aloe vera, preservatives, vitamins and minerals such as riboflavin and fragrance.

The dispersing agent can be any of those employed in the art that does not adversely affect the photochromic monomers, but based on considerations of availability and cost, ethanol is the preferred dispersing agent.

Second Embodiment—Composition Containing Pigment Particles

The photochromic hair coloring composition of the present invention may be in the form of particles containing photochromic material. In practice it has been found, for reasons of color change stability in environmental conditions, that pigment particles are preferred over monomeric pigments. These particles can be adhered to hair using a binder composition or anti-hair antibodies. The adherent is preferably also a suspending agent, which aides in dispersing and suspending the photochromic pigment in the photochromic composition prior to application to hair, and which imparts adherence of the photochromic pigment to hair after application to hair (i.e., acts as a “glue” for the photochromic pigment particles, without adversely effecting the luster and silkiness of hair).

Examples of patents teaching photochromic particles considered safe for cosmetics include U.S. Pat. Nos. 5,176,905, 5,762,913, and 4,929,693 discussed above.

U.S. Pat. No. 5,597,386 (Igarashi, et al.) entitled Hair coloring composition comprising anti-hair antibodies immobilized on coloring materials, and hair coloring methods teaches a hair dye consisting of an anti-hair antibody immobilized on a high bulky coloring material. The coloring material may be a coloring substance such as an inorganic pigment, water-soluble coloring matter, or water-insoluble coloring matter, or a composite of the coloring substance and a macromolecular carrier. The formulation of the present invention differs in the discovery of the possibility of utilization of photochromic pigments in the pigment particles. Since such pigment particles provide a good and strong coloring capability specific to hair, the hair dye will neither stain the skin nor cause skin irritation. Further, the touch and feel of the hair will be improved.

Anti-hair antibodies are antibodies having an immunoactivity to human hair, and possess a molecular diameter of up to 10 nm. These include antibodies against various components that constitute the hair to be colored. These antibodies can be obtained by immunizing animals using as antigens hair keratin protein, hair cuticle protein, hair matrix protein or fragments thereof. Further, these antibodies can be obtained by using animal nails, body hair, feathers, or extracts and fragments thereof as antigens. In view of differences among species, human hair from either the head or the body is preferably used, and especially various components that constitute human hair are most preferable.

Those animals to be immunized can be selected from domestic animals that include bovines, horses, sheep, goats, rabbits, chickens and the like.

Antibodies can be isolated from milk or colostrum, serum, or egg yolk produced by these animals. The antibodies obtained from bovine milk or colostrum, and egg yolk are preferable because they can be produced in a large quantity.

Hair cosmetics or hair care products can contain hair dyes in an amount of 0.01-80% based on the total weight.

The macromolecular carriers can be divided into two categories: water-insoluble macromolecular carriers and water-soluble macromolecular carriers. Water-insoluble macromolecular carriers are advantageous in improvements in tensity, resiliency, tautness, slipperiness, collectivity and feel of hair, and in particular synthetic polymers are preferable because of significant improvement in tensity, resiliency and tautness of hair. Water-insoluble proteins provide suppleness, water-insoluble polysaccharides can provide humid feeling, and liposomes can provide gloss. On the other hand, water-soluble macromolecular carriers are advantageous in improving humid feeling, smoothness, flexibility, slipperiness and feel of hair.

Water-insoluble macromolecular carriers include synthetic polymers (polychlorostyrene, polychloromethyl styrene, polyacrylic acid, polymethacrylic acid, polymaleic acid, polystyrene sulfonic acid, etc., preferably polystyrene and derivatives), water-insoluble proteins (fibroin, gelatin, collagen, etc.), water-insoluble polysaccharides (cross-linked agarose, cross-linked dextran, chitin, etc.) liposomes (made of phospholipids such as phosphatidylcholine, phosphatidylserine, phosphatidyl ethanolamine, etc.) and the like.

The ratio of the water-insoluble macromolecular carriers to the antibody raw materials in general is 0.01-100 mg of the antibody raw material immobilized on 1 g of the water-insoluble macromolecular carrier. Less than 0.01 mg is likely to provide insufficient coloring capability. On the other hand, with the addition of more than 100 mg, no substantial increase in coloring capability is attained.

As to the water-soluble macromolecular carriers, either natural or synthetic ones can be used. Natural polymers include polysaccharides (starch, amylose, amylopectin, pectin, carrageenan, mannan, galactan, sodium alginate, tragacanth gum, gum arabic, dextran, pullulan, Curdlan, levan, glucan, succinoglucan, Xanthane Rubber, hyaluronic acid, chondroitin sulfate, etc.) and proteins of natural origin (glue, gelatin, casein, collagen, fibroin, etc.) and derivatives thereof. Synthetic polymers include polyvinylalcohol, carboxyvinyl polymers, polysodiumacrylate, polyvinylpyrrolidone, polyethyleneoxide, polylysine, polyglutamic acid, polyaspartic acid and the like.

In order to support coloring substances on water-insoluble macromolecular carriers or water-soluble macromolecular carriers, physical and chemical methods are utilized. The physical methods include an adsorption method, an internal addition method, an inclusion method, and a polymerization method.

The chemical binding also can immobilize the coloring substances if functional groups exist in the water-insoluble or water-soluble macromolecular carriers. In order to immobilize antibodies on water-insoluble or water-soluble macromolecular carriers, physical adsorption and chemical binding are utilized.

The ratio of the water-soluble macromolecules to the antibody raw materials depends on the water-soluble macromolecule to be used. However, in general, 0.01 mg-1 g of the antibody raw material is preferably immobilized on 1 g of the water-soluble macromolecule.

Particles preferably have uniform grain size, preferably in the range of an average diameter of 0.001-100 μm, and more preferably 0.001-1 μm. The most preferred range is 0.05-0.7 μm with which size a high hair dyeing efficiency can be accomplished.

Applicators

The hair colorant composition of the first or second embodiments discussed above may be formulated as flowable compositions such as solutions, creams, lotions, gels or emulsions, and as such may be applied using an applicator similar to a mascara applicator. Other examples of applicators include those disclosed in the following patents.

U.S. Pat. No. 5,937,866 (Magharehi) entitled Hair dye applicator teaches a hair dye applicator comprising a container having a variety of interchangeable components through which hair dye is discharged onto the hair. A first interchangeable component has a roller ball and is ideal for use when distribution of hair dye along a part at the root of the hair is desired. A second interchangeable component has a plurality of flexible brushes, and is ideal for distribution of hair dye throughout the hair and along the hair shafts. A third interchangeable component has a plurality of rigid combs, and is ideal for overall distribution of hair dye in coarse or thick hair. Both second and third interchangeable components contain a moveable disc which allows a user to control the rate of discharge of hair dye from the hair dye applicator.

U.S. Pat. No. 5,937,865 (Dhaliwal) entitled Hair coloring applicator teaches a hair coloring applicator including a hair dye tube adapted for holding a quantity of hair coloring. A comb portion is provided that is adapted for coupling with open port of the hair dye tube. The housing has a plurality of hollow bristles extending downwardly from the lower wall thereof. The hollow bristles are in communication with the hollow interior of the housing. Each of the hollow bristles have a plurality of pores therein in a spaced relationship whereby hair coloring from the hair dye tube is dispensed through the open port into the housing through the feed duct and out through the pores of the plurality of bristles for application on strands of a user's hair.

U.S. Pat. No. 5,931,168 (Abercrombie, et al.) entitled Applicator for coloring hair or fibers and methods for making and using same teaches an applicator for transferring color-altering material from a substrate to hair or fibers, and methods for making and using the same. A color-altering dye material, which is soluble in water and water-activated, is adhered to one or both portions of the applicator. The applicator is flexible, thin, and is made of a material such as a foil, a paper composition or a synthetic polymer which is conformable to the shape of the human hand. Use of the applicator requires wetting the hairs or fibers, and then applying the substrate to the hairs or fibers.

The hair colorant composition of the present invention may contain, in addition to the photochromic components, other coloring components as well as components commonly associated with the formulation of solutions, creams, lotions, gels or emulsions, and the like. For example, components such as wetting agents or emulsifying agents from the categories of anionic or non-ionic surfactants, such as sulfates of fatty alcohols, alkanolamides of fatty alcohols, alkyl sulfonates, alkylbenzene sulfonates, oxyethylated fatty alcohols, oxyethylated nonylphenols. Additionally, thickeners, such a fatty alcohols, starch, cellulose derivatives, paraffin oil and fatty acids, as well as hair-care substances, such as lanolin derivatives, cholesterol and pantothenic acid, may be formulated into the compositions of the invention.

In addition, the hair colorant compositions may optionally contain conventionally-used adjuvants and cosmetic additives, or mixtures thereof, to achieve the final formulations. Examples of such additives include, but are not limited to, anti-oxidants, e.g. ascorbic acid, erythorbic acid, or sodium sulfite, to inhibit premature oxidizing; oxidizing agents, fragrances and/or perfume oils; chelating agents; emulsifiers; coloring agents; thickeners; organic solvents; opacifying agents; dispersing agents; sequestering agents; hair-care substances; humectants; anti-microbials; acidifying agents and others. The list of optional ingredients is not intended as limiting. Other suitable adjuvants for inclusion in the hair dye compositions of the invention are disclosed, for example, in Zviak, The Science of Hair Care (1986) and in Balsam and Sagarin, Cosmetics: Science and Technology, Vol. 2, Second Edition, (1972).

Fragrance

If added, substances used to produce a desired fragrance may be any one or more of those which are commonly used by those skilled in the art of toiletry fragrance chemistry or perfumery, some of which are listed in the following texts: Robert R. Calkin, J. Stephan Jellinek, Perfumery, Practice and Principle, John Wiley and Sons, Inc., New York, 1994; Rudiger Hall, Dieter Klemme, Jurgen Nienhaus, Guide to Fragrance Ingredients, H&R Edition, R. Gross & Co. Publishing, Hamburg, 1985; Julia Muller, The H&R Book of Perfume, H&R Edition, Johnson Publications, Ltd., London, 1984; Fragrance Guide-Feminine Notes, Masculine Notes, H&R Edition, R. Gross & Co. Publishing, Hamburg, 1985 which are incorporated by reference herein.

### EXAMPLE 1

Product: Color Changing Hair Dye for use with Mascara Applicator

| Item | % | Ingredient | Supplier |
|---|---|---|---|
| 1. | 50.1 | Deionized Water | |
| 2. | 1.0 | Germaben II | ISP |
| 3. | 0.3 | Carbopol Ultrez 10 | BF Goodrich |
| 4. | 2.0 | SF 1188A | GE Silicones |
| 5. | 25.0 | SD 39C | |
| 6. | 20.0 | Photochromic Pigment 1 | |
| 7. | 1.0 | Emulsifier/Solubilizer | Dragoco |
| | | 2/014160 | |
| 8. | 0.6 | Triethanolamine 50% | |
| | 100.00 | Total | |

Photochromic pigment 1: trademark: Photopia Yellow, Matsui Shikiso Chemical Co., Ltd.; a naphthopyran photochromic compound, a synthetic resin and a plasticizer all microencapsulated in a thermosetting resin)

Formula Preparation

In Example 1 100 ml of a color changing co position for use with a mascara applicator were produced. Thus, the above percentages are simply converted into ml, with each percent converting to one ml. First, in a 500 ml vessel, ingredients 1 and 2 were mixed. Then, Carbopol was dispersed into the solution making sure that it is free from lumps. In a separate vessel ingredients 4-7 were pre-mixed by homogenizing for 1 minute, and then this mixture was added to the mixture of ingredients 1 and 2. Finally, ingredient 8 was mixed in until homogenous.

A mascara brush was used to apply the composition to human hair. The composition was relatively colorless in a room not exposed to direct sunlight, but turned deep yellow when exposed to direct sunlight.

### EXAMPLE 2

Product: Color Changing Hair Coloring Composition for use with
Mascara Applicator

| Item | % | Ingredient | Supplier |
|---|---|---|---|
| 1. | 51.9 | Deionized Water | |
| 2. | 0.2 | Germall II | ISP |
| | | DIAZOLIDNYL UREA | |
| | | Carbopol Ultrez 10 | B. F. |
| 3. | 0.3 | CARBOMER | GOODRICH |
| 4. | 2.0 | SF 1188A | GE |
| | | SILICONE POLYETHER COPOLYMER | Silicones |
| 5. | 25.0 | SD 39 C alcohol | |
| 6. | 20.0 | Photochromic Pigment Slurry 2 | Clark R & D |
| 7. | 0.6 | Triethanolamine (50% soln) | |
| | 100.00 | Total | |

Photochromic Pigment Slurry 2: A microencapsulated photochromic material [1 part by weight of 1,3,3-trimethylspiro[indoline-2,3′-(3H)naphtho(2,1-b) (1,4)-oxazine] (an organic photochromic compound) and a bis(1,2,2,6,6-pentamethyl-4-piperidinyl) sebacate (a hindered amine compound) microencapsulated in 5 parts by weight of epoxy resin.

Formula Preparation

As in Example 1, 100 ml of product were prepared. First, deionized water was weigned in a beaker. Then, ingredient #2 was added with mixing. Slowly, Carbopol is dispersed making sure that the solution was free from lumps. Next, ingredient #4 was added and mixing was continued.

In a separate beaker, alcohol and pigment were pre-mixed, and then slowly added to the first mixture. Mixing was continued until the batch was homogenous. Then, the batch was neutralized with the triethanolamine solution.

The formulation was applied to human hair using a mascara brush. The formulation was clear-to-white in a room not exposed to direct sunlight, and turned deep blue when exposed to direct sunlight.

Although this invention has been described in its preferred form with a certain degree of particularity with respect to a formulation for coloring hair, it is understood that the present disclosure of the preferred form has been made only by way of example, and that numerous changes in the details of structures and composition of the product may be resorted to without departing from the spirit and scope of the invention.

What is claimed is:

1. A photochromic hair coloring composition comprising photochromic pigment, a suspending agent, and sufficient rapid-drying dispersant to form a photochromic slurry, wherein said suspending agent promotes bonding of photochromic pigment to hair.

2. A photochromic hair coloring composition as in claim 1, wherein said suspending agent is selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives.

3. A photochromic hair coloring composition as in claim 2, wherein said polyacrylic acid is a polymer of acrylic acid cross-linked with a polyfunctional agent.

4. A photochromic hair coloring composition as in claim 1, wherein said photochromic monomers are selected from spiropyran compounds, spiroxazine compounds, azobenzene compounds, thioindigo compounds, dithizone metal complex compounds, fulgide compounds, dihydropyrene compounds, spirothiopyran compounds, 1,4-2H-oxazine, triphenylmethane compounds, viologen compounds, naphthopyran compounds, and benzopyran compounds.

5. A photochromic hair coloring composition as in claim 1, wherein said photochromic monomers are selected from spiropyran compounds, spirooxazine compounds, fulgide compounds, naphthopyran compounds and benzopyran compounds.

6. A photochromic hair coloring composition as in claim 1, wherein said dispersant is ethanol.

7. A photochromic hair coloring composition comprising photochromic particles and a binder.

8. A photochromic hair coloring composition as in claim 7, wherein said binder is a carbopol gel matrix, and wherein said composition further comprises sufficient rapid-drying dispersant to form a photochromic slurry.

9. A photochromic hair coloring composition comprising photochromic particles, wherein anti-hair antibodies are immobilized on said photochromic pigment particles.

10. A method for selectively rendering hair photochromic, said method comprising:
(a) applying to a brush an amount of a liquid photochromic hair coloring composition, and
(b) selectively applying said composition to hair.

11. A method as in claim 10, wherein said steps (a) and (b) are repeated two or more times with different photochromic hair coloring compositions and to different selective areas of the hair.

12. A method as in claim 10, further comprising bleaching hair to a lighter color prior to treating with said photochromic hair coloring composition.
